(19) United States
(12) Patent Application Publication
(10) Pub. No.: US 2024/0408290 A1
(43) Pub. Date: Dec. 12, 2024

(54) CONFIGURING AN ARRANGEMENT TO GENERATE TREATMENT FLUID FOR RENAL REPLACEMENT THERAPY

(71) Applicants: BAXTER INTERNATIONAL INC., Deerfield, IL (US); BAXTER HEALTHCARE SA, Glattpark (Opfikon) (CH)

(72) Inventors: Jonas FORS, Malmö (SE); Thomas HERTZ, Lund (SE)

(21) Appl. No.: 18/698,681

(22) PCT Filed: Sep. 15, 2022
(86) PCT No.: PCT/EP2022/075676

§ 371 Date: Apr. 4, 2024

(30) Foreign Application Priority Data
Oct. 5, 2021 (SE) 2151218-1

(51) Int. Cl. A61M 1/16; A61M 1/28

(52) U.S. Cl. CPC A61M 1/1666; A61M 1/287; A61M 2205/18; A61M 2205/502

(57) ABSTRACT

A system comprises a fluid generation arrangement (FGA), a user interface (UI), and a control unit. The FGA is operable to mix one or more concentrates with water to generate a treatment fluid for use in renal replacement therapy. The control unit is connected to the UI and arranged to configure the FGA by a computer-implemented method. In the method, the control unit receives, from the UI, a candidate set value of a selected component of the treatment fluid, calculates, for the candidate set value, a calculated composition of the treatment fluid, and displays, on the UI, a respective concentration value of one or more components other than the selected component in the calculated composition. By the system, a caretaker is made aware of consequential changes in the composition of the treatment fluid, in addition the selected component.

### TECHNICAL FIELD

The present disclosure relates to the field of renal replacement therapy and in particular to on-line generation of a treatment fluid for use in such therapy.

### BACKGROUND ART

Renal replacement therapy (RRT) is a therapy that replaces the normal blood-filtering function of the kidneys. It is used when the kidneys are not working well, which is known as kidney failure and includes acute kidney injury and chronic kidney disease. RRT involves removal of solutes from the blood of a patient suffering from kidney failure, for example by hemodialysis (HD) or peritoneal dialysis (PD). Often, RRT is performed by use of a machine.

In RRT, one or more treatment fluids of specific composition are used for treatment of the patient. Over time, RRT consumes large quantities of treatment fluid. In some modalities of RRT, ready-made treatment fluid is delivered in prefilled bags to the point of care. For example, conventional PD is performed by use of prefilled bags. In HD, different types of machines are used for treatment of patients with acute kidney injury (AKI) and patients with chronic kidney disease (CKD). Machines for treatment of patients with AKI are generally configured to use prefilled bags of ready-made treatment fluid, whereas machines for treatment of patients with CKD generally have integrated capability to generate treatment fluid on-demand by mixing one or more concentrates with water, so-called on-line fluid generation. Recently, PD machines with integrated capability of on-line fluid generation have been proposed.

It is envisioned that on-line fluid generation will at least to some extent substitute prefilled bags in all types of RRT in the future, for example in view of environmental concerns related to the transportation of prefilled bags and issues related to the handling of heavy prefilled bags. On-line fluid generation has the further advantage of allowing a caretaker to adjust the composition of the treatment fluid to improve the treatment of the patient, without changing the prefilled bags.

One limitation of on-line fluid generation is that a concentrate, for logistical reasons, may contain a plurality of substances. Thus, a deliberate change in the concentration of one substance in the treatment fluid may result in a change in one or more other substances, depending on the type of concentrate(s) that are used. A caretaker that initiates a desired concentration change of one substance in a treatment fluid may overlook or even be unaware of an inherent concentration change of another substance.

This problem is applicable to all types of RRT that use on-line fluid generation today and in the future. As noted, future HD machines for treatment of AKI may rely on on-line fluid generation instead of or in addition to ready-made fluids. These HD machines are primarily used in intensive care units (ICUs), where caretakers have to handle many different types of life-saving equipment and are not experts on RRT. It may thus be particularly difficult for ICU staff to make full use of the capabilities offered by on-line fluid generation.

### SUMMARY

It is an objective to at least partly overcome one or more limitations of the prior art.

A further objective is to facilitate the use of on-line fluid generation in RRT.

Another objective is to improve patient safety in RRT using treatment fluids generated by mixing one or more concentrates with water.

One or more of these objectives, as well as further objectives that may appear from the description below, are at least partly achieved by a system, a computer-implemented method and a computer-readable medium according to the independent claims, embodiments thereof being defined by the dependent claims.

A first aspect is a system comprising a fluid generation arrangement, which is operable to mix one or more concentrates with water to generate a treatment fluid for use in renal replacement therapy, a user interface, and a control unit, which is connected to the user interface and arranged to configure the fluid generation arrangement. The control unit is configured to: receive, from the user interface, a candidate set value of a selected component of the treatment fluid; calculate, for the candidate set value, a calculated composition of the treatment fluid; and display, on the user interface, a respective concentration value of one or more components other than the selected component in the calculated composition.

The system of the first aspect allows a user, for example a caretaker, to change a set value for a fluid generation arrangement that affects a selected component in the resulting treatment fluid, while ensuring that the user is also made aware of consequential changes to other components in the treatment fluid, for example as a result of a concentrate containing more than one component. The first aspect thereby facilitates the use of the on-line capability of the fluid generation arrangement without compromising patient safety.

The system of the first aspect is thus configured to display a consequential property of the treatment fluid, if the treatment fluid were to be generated based on the candidate set value. The cognitive content of the property presented to the operator, i.e. the one or more concentration values, relates to a potential internal state of the system and enables the user to properly operate the system. This potential internal state may dynamically change depending on the candidate set value entered by the user and is automatically detected as the system calculates the calculated composition resulting from the candidate set value. The display of the concentration value(s) may prompt the user to interact with the system, specifically to decide if the property of the treatment fluid is deemed suitable for the patient and the RRT to be performed and provide the confirmation signal if the property is deemed suitable. The skilled person also realizes that the manner in which the property is presented, i.e. upon entry of the candidate set value, assists the user in performing a technical task by means of a guided human-machine interaction process.

In some embodiments, the control unit is further configured to configure, upon receipt of a confirmation signal from the user interface, the fluid generation arrangement to generate the treatment fluid in accordance with the calculated composition.

In some embodiments, the control unit is operable to receive the candidate set value, calculate the calculated composition, and display the respective concentration value while the fluid generation arrangement is operated to generate the treatment fluid with a current composition which differs from the calculated composition, and wherein the control unit is configured to, in absence of the confirmation signal, maintain the fluid generation arrangement in operation to generate the treatment fluid in accordance with the current composition.

In some embodiments, the one or more concentrates comprises a combination of an acid concentrate and a bicarbonate concentrate.

In some embodiments, the selected component comprises one of sodium, bicarbonate or potassium.

In some embodiments, the one or more components other than the selected component comprises one or more electrolytes.

In some embodiments, the one or more electrolytes is one or more of magnesium, potassium, calcium, sodium, chloride, phosphate, bicarbonate, lactate, acetate, citrate.

In some embodiments, the one or more components other than the selected component comprises an osmotic agent.

In some embodiments, the one or more components other than the selected component comprises glucose.

In some embodiments, the control unit is further configured to: obtain nominal composition data of the one or more concentrates, wherein the control unit is configured to calculate the calculated composition as a function of the nominal composition data, so that the candidate set value is achieved in the calculated composition.

In some embodiments, the control unit is further configured to: evaluate concentrations of components in the calculated composition in relation to a respective concentration range, and display a warning message on the user interface if at least one of the components has a concentration that deviates from the respective concentration range.

In some embodiments, the control unit is configured to display the warning message so as to indicate the at least one of the components on the user interface.

In some embodiments, the concentrations of components comprise nominal concentrations, and the control unit is configured to calculate the nominal concentrations based on a nominal composition of a respective concentrate among the one or more concentrates.

In some embodiments, the concentrations of components comprise at least one of a maximum concentration or a minimum concentration of a respective component in the calculated composition, and the control unit is configured to calculate the at least one of a maximum concentration or a minimum concentration based on an allowable deviation from a nominal concentration in the treatment fluid, and the control unit is further configured to display the warning message if the at least one of a maximum concentration or a minimum concentration deviates from the concentration range.

In some embodiments, the system further comprises a treatment arrangement for renal replacement therapy, and the control unit is further configured to operate the fluid generation arrangement to generate the dialysis fluid in accordance with the calculated composition, and to operate the treatment arrangement to perform the renal replacement therapy by use of the treatment fluid generated by the fluid generation arrangement.

In some embodiments, the treatment arrangement is configured to perform extracorporeal blood treatment.

In some embodiments, the treatment arrangement is configured to perform treatment of acute kidney injury, AKI.

In some embodiments, the treatment arrangement is configured to perform peritoneal dialysis.

In some embodiments, the control unit is configured to automatically, upon receiving the candidate set value from the user interface, configure the fluid generation arrangement to generate the treatment fluid in accordance with the calculated composition.

A second aspect is a computer-implemented method of configuring a fluid generation arrangement, which is operable to mix one or more concentrates with water to generate a treatment fluid. The method comprises: receiving, from a user interface, a candidate set value of a selected component of the treatment fluid; calculating, for the candidate set value, a calculated composition of the treatment fluid; and displaying, on the user interface, a respective concentration value of one or more components other than the selected component in the calculated composition.

The embodiments of the first aspect may be adapted as embodiments of the second aspect as well. For example, in some embodiments, the method further comprises: configuring, upon receipt of a confirmation signal from the user interface, the fluid generation arrangement to generate the treatment fluid in accordance with the calculated composition. In alternative embodiments, the method further comprises: configuring, upon said receiving the candidate set value, the fluid generation arrangement to generate the treatment fluid in accordance with the calculated composition.

A third aspect is a computer-readable medium comprising computer instructions which, when executed by a processor, cause the processor to perform the method of the second aspect and any of its embodiments.

The second and third aspects share technical advantages with the first aspect.

Still other objectives, aspects and advantages, as well as features and embodiments, may appear from the following detailed description, from the attached claims as well as from the drawings.

### BRIEF DESCRIPTION OF THE DRAWINGS

FIG. 1 is a block diagram of an example system comprising a fluid generation arrangement and a control unit.

FIGS. 2A-2C are flowcharts of example methods or procedures that may be performed by the control unit in FIG. 1.

FIGS. 3A-3C are schematic examples of messages displayed to a user by the method in FIG. 2A.

FIG. 4 is a block diagram of an example fluid circuit for mixing concentrates with water into a treatment fluid.

FIG. 5 is a schematic diagram of an example extracorporeal blood circuit for RRT.

### DETAILED DESCRIPTION OF EXAMPLE EMBODIMENTS

Embodiments will now be described more fully hereinafter with reference to the accompanying drawings, in which some, but not all, embodiments are shown. Indeed, the subject of the present disclosure may be embodied in many different forms and should not be construed as limited to the embodiments set forth herein; rather, these embodiments are provided so that this disclosure may satisfy applicable legal requirements. Like numbers refer to like elements throughout.

Also, it will be understood that, where possible, any of the advantages, features, functions, devices, and/or operational aspects of any of the embodiments described and/or contemplated herein may be included in any of the other embodiments described and/or contemplated herein, and/or vice versa. In addition, where possible, any terms expressed in the singular form herein are meant to also include the plural form and/or vice versa, unless explicitly stated otherwise. As used herein, “at least one” shall mean “one or more” and these phrases are intended to be interchangeable. Accordingly, the terms “a” and/or “an” shall mean “at least one” or “one or more,” even though the phrase “one or more” or “at least one” is also used herein. As used herein, except where the context requires otherwise owing to express language or necessary implication, the word “comprise” or variations such as “comprises” or “comprising” is used in an inclusive sense, that is, to specify the presence of the stated features but not to preclude the presence or addition of further features in various embodiments.

It will furthermore be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing the scope of the present disclosure. As used herein, the terms “multiple”, “plural” and “plurality” are intended to imply provision of two or more elements. The term “and/or” includes any and all combinations of one or more of the associated listed elements.

Well-known functions or structures may not be described in detail for brevity and/or clarity. Unless otherwise defined, all terms (including technical and scientific terms) used herein have the same meaning as commonly understood by one of ordinary skill in the art to which this disclosure belongs.

As used herein, a “concentrate” is a substance that contains one or more compounds at a concentration that is higher than at the final use of the substance. A concentrate may be in the form a liquid or a powder. A concentrate is capable of being diluted, if a liquid, or dissolved, if a powder, in a solvent. In the context of the present description, the solvent is water. Before or after being diluted/dissolved in the solvent, the concentrate may be mixed with one or more other concentrates, which also may be in the form of a liquid or a powder.

As used herein, RRT refers to any machine-based therapy that replaces the normal blood-filtering function of the kidneys. RRT includes therapies involving extracorporeal treatment of the blood, including but not limited to hemodialysis (HD), hemodiafiltration (HDF) and hemofiltration (HF). RRT also includes peritoneal dialysis (PD), in any modality. Extracorporeal blood treatment is commonly differentiated by the origin of the kidney failure into “acute blood treatment”, for patients with AKI, and “chronic blood treatment”, for patients with CKD. Generally, acute blood treatment differs from chronic blood treatment by using a prolonged treatment time and lower flow rates of blood and treatment fluid. Chronic blood treatment is typically performed in intermittent sessions, for example with a duration of 3-5 hours, for example 2-3 times a week, whereas acute blood treatment may be performed continuously (24-hour treatment) or semi-continuously, for example daily with a duration of 6-12 hours or more. Non-limiting examples of acute treatments include CRRT (Continuous Renal Replacement Therapy), CVVH (continuous veno-venous hemofiltration), CVVHD (continuous veno-venous hemodialysis), CVVHDF (continuous veno-venous hemodiafiltration), SLEF (slow extended hemofiltration), SLED (sustained low-efficiency dialysis), and PIRRT (Prolonged Intermittent Renal Replacement Therapy).

As used herein, a “treatment fluid” refers to any liquid solution that may be administered to the patient as part of an RRT treatment. The treatment fluid may be a dialysis fluid, which is administered to a patient in extracorporeal blood treatment or peritoneal dialysis, to bring about a cleaning of the blood of the patient. The treatment fluid may also be a so-called replacement fluid or substitution fluid, which is administered directly into the blood of the patient in HDF or HF.

As used herein, a “component” of a treatment fluid refers to any compound in a treatment fluid that is of relevance to the RRT or the well-being of the patient. The component may be an electrolyte or a nonelectrolyte.

As used herein, “electrolytes” refer to ions in the treatment fluid, where an ion is a particle, atom or molecule with a net electrical charge.

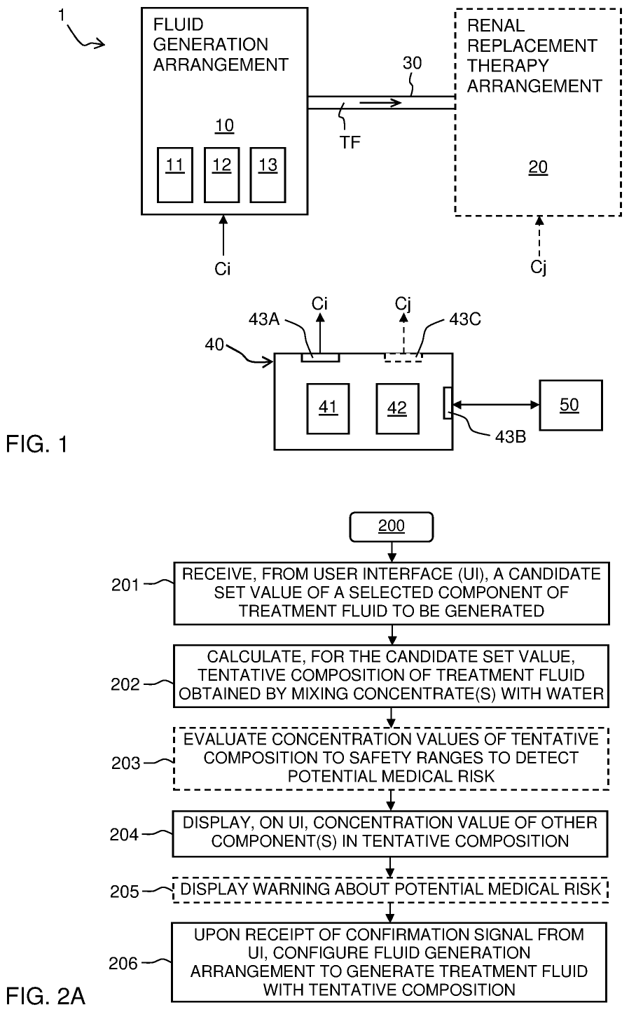

FIG. 1 shows an example system 1 which will be used to describe some embodiments. The system 1 comprises a fluid generation arrangement (FGA) 10, which is operable to mix two different concentrates with water to produce a treatment fluid. The water is schematically represented by reference numeral 11, and the concentrates are schematically represented by reference numerals 12, 13. The resulting treatment fluid TF is output by the FGA 10 on a fluid line 30 for use by an RRT arrangement 20, which may or may not be part of the system 1, as indicated by dashed lines. The RRT arrangement 20 is configured to perform a conventional RRT treatment by use of the treatment fluid TF. The system 1 further includes a control unit or controller 40, which is configured to provide one or more control signals Ci for the FGA 10. By the control signal(s) Ci, the control unit 40 causes the FGA 10 to generate the treatment fluid TF with a desired composition. In one conceivable implementation, the FGA 10 has a local controller (not shown) that controls the operation of the FGA 10. In such an implementation, the control signal(s) Ci may include configuration data that allows the local controller to operate the FGA 10 to produce the treatment fluid TF with the desired composition. In another conceivable implementation, the control unit 40 directly controls the operation of the FGA 10 by providing a plurality of control signals Ci to various operative elements in the FGA 10, such as pumps, valves, etc. Although not shown in FIG. 1, the control unit 40 may be configured to receive one or more output signals of the FGA 10, for example from one or more sensors. An example of an FGA 10 will be described below with reference to FIG. 4.

If the system 1 includes the RRT arrangement 20, the control unit 40 may also be configured to provide one or more control signals Cj for the RRT arrangement 20. By the control signal(s) Cj, the control unit 40 causes the RRT arrangement 20 to perform an RRT treatment by use of the treatment fluid. The RRT arrangement 20 may be operated to consume the treatment fluid at a rate that matches the output rate of treatment fluid from the FGA 10, or to buffer the treatment fluid. The control unit 40 may, by the control signal(s) Cj, provide configuration data for a local processor (not shown) in the RRT arrangement 20 or directly control various operative elements in the RRT arrangement 20, such as pumps, valves, etc. Although not shown in FIG. 1, the control unit 40 may be configured to receive one or more output signals of the RRT arrangement 20, for example from one or more sensors. An example of an RRT arrangement 20 will be described below with reference to FIG. 5.

The control device 40 comprises a processor 41 and computer memory 42. A control program may be stored in the memory 42 and executed by the processor 41 to perform any of the methods, procedures, or functions as described herein. The control program may be supplied to the control device 40 on a computer-readable medium, which may be a tangible (non-transitory) product (e.g., magnetic medium, optical disk, read-only memory, flash memory, etc.) or a propagating signal. In the illustrated example, the control device 40 comprises a signal interface 43A for providing the control signal(s) Ci to the FGA 10. The control device 40 also comprises an input/output (I/O) interface 43B for connection to a user interface (UI) 50. The term “user interface” is intended to include any and all devices that are capable of performing guided human-machine interaction comprising display of information and receipt of input. Thus, the UI 50 may comprise a combination of a display device and data entry hardware. The data entry hardware may include one or more of a keyboard, keypad, computer mouse, control buttons, touch panel, microphone and voice control functionality, camera and gesture control functionality, etc. In one implementation, the UI 50 is or comprises a touch-sensitive display, also known as touch screen. The I/O interface 43B may be operable to output a signal that controls the display function of the UI 50 and to input a response signal indicative of entries made by the user via the UI 50. The UI 50 may or may not be integrated with the control unit 40. As further indicated in FIG. 1, the control device may comprise a further signal interface 43C for providing the control signal(s) Cj to the RRT arrangement 20. Each of the interfaces 43A, 43B, 43C may be configured for wired or wireless data transmission in accordance with any standardized or proprietary protocol. It may be noted that the interfaces 43A, 43B and 43C (if present) may be implemented by a single interface device.

The system 1 as depicted in FIG. 1, including the RRT arrangement 20, may be integrated into a dialysis machine. In an alternative, the system 1 without the RRT arrangement 20 may be integrated into a fluid preparation device. In a further alternative, the RRT arrangement 20 and the control unit 40 are integrated into a dialysis machine, whereas the FGA 10 is physically separate from the dialysis machine. In another alternative, the control unit 40 may be physically separate from both the FGA 10 and the RRT arrangement 20, for example a conventional computer.

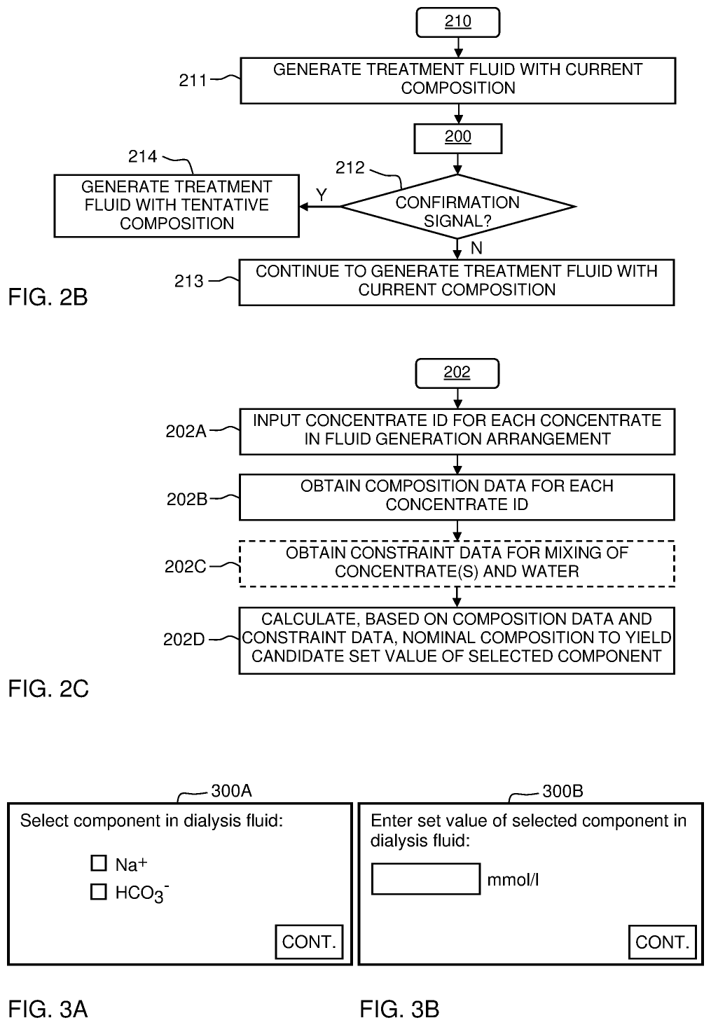

FIG. 2A is a flowchart of an example method 200 that may be performed by the control unit 40 in FIG. 1. Dashed lines indicate optional steps. The method 200 aims at assisting a user (for example, a caretaker) that wishes to generate a treatment fluid with a specific concentration of a selected component. As understood from the discussion in the Background section, at least one of the concentrates 12, 13 used by the FGA 10 in FIG. 1 may be a multi-component concentrate that contributes with a plurality of components to the treatment fluid. If the selected component originates from such a multi-component concentrate, the concentration of the selected component cannot be adjusted independently of additional components in the multi-component concentrate. It is also conceivable that the selected component originates from two or more concentrates and/or that another component of the treatment fluid originates from two or more concentrates. This interdependence between components may make it quite difficult, even for an experienced caretaker, to grasp how a modified concentration of a selected component will affect the concentrations of other components in the treatment fluid. This difficultly is further aggravated if the clinic has a practice of using different combinations of concentrates in its FGAs from time to time, or if different FGAs within the clinic use different combinations of concentrates.

In step 201, the control unit 40 receives from the UI 50 a candidate set value of a selected component in the treatment fluid to be generated. The candidate set value may be a desired concentration of the selected component. Step 201 may be the result of the caretaker selecting a component and entering the candidate set value via the UI 50.

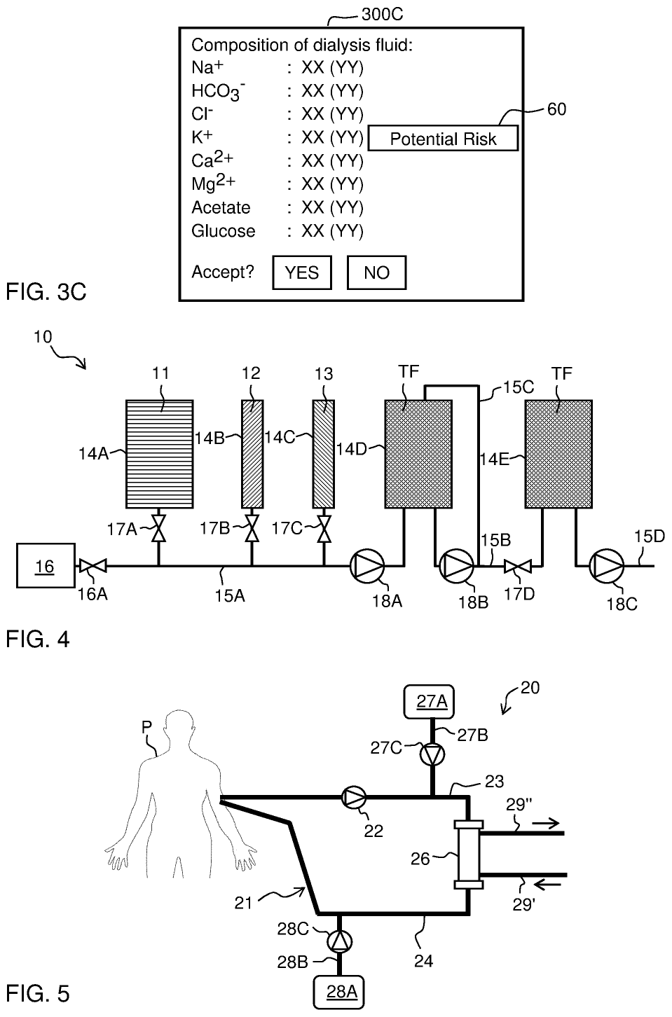

FIG. 3A shows an example of a screen image 300A, which may be displayed on the UI 50 after a user has commanded the control unit 40 to enable modification of the treatment fluid. In the illustrated example, screen image 300A prompts the user to select between two components in the treatment fluid, specifically sodium (Na⁺) or bicarbonate (HCO₃⁻). It may be relevant for a caretaker to adjust these specific components in HD treatment. The sodium concentration in dialysis fluid for HD treatment may be adjusted in view of the patient's pre-dialysis serum sodium concentration, for example to improve treatment tolerability and prevent unnecessary sodium loading. In order to correct metabolic acidemia, patients on HD treatment require replenishment of bicarbonate via diffusion from dialysis fluid. The concentration of bicarbonate in the dialysis fluid is commonly individualized to the patient to ensure that the serum concentration of bicarbonate is within physiologically acceptable limits. However, it is to be understood that the user may be allowed to adjust further and/or other components in the treatment fluid, depending on modality and treatment fluid. It is also conceivable that the user is allowed to select more than one component.

When the user has selected a component in screen image 300A and activated the continue button (CONT.), the UI 50 may be operated to present screen image 300B of FIG. 3B. The screen image 300B prompts the user to enter a set value for the concentration of the selected component in a dedicated field. Although not shown, the screen image 300B may present a current value of the selected component, if step 201 is performed during on-going fluid generation. When the user has entered the candidate set value and activated the continue button (CONT.), step 201 is completed by the control unit 40.

In step 202, the control unit 40 calculates, for the candidate set value, a tentative composition of the treatment fluid. The tentative composition is also denoted “calculated composition” herein. The tentative composition is calculated to at least approximately match the candidate set value. In other words, the tentative composition includes the selected component at a concentration that matches the candidate set value. The tentative composition also includes concentration values of one or more other components in the treatment fluid. As used herein, “other components” refer to components other than the selected component in the treatment fluid to be generated.

In step 204, the control unit 40 displays, on the UI 50, a respective concentration value of the other component(s) in accordance with the tentative composition that is calculated in step 202. Thereby, the caretaker will be made aware of the impact of the modified concentration of the selected component on the other component(s) in the treatment fluid. The control unit 40 may also display the concentration value of the selected component. An example is shown in FIG. 3C, which shows a screen image 300C that may be displayed on the UI 50 in step 204. In the illustrated example, screen image 300C lists concentration values (indicated by XX) of the selected component (sodium or bicarbonate in FIG. 3A) and a plurality of other components, including chloride (Cl⁻), potassium (K⁺), calcium (Ca²⁺), magnesium (Mg²⁺), acetate and glucose. If the method 200 is performed during an on-going RRT, the screen image 300C may also show the current concentration values of the respective component (indicated by YY), to further assist the caretaker in taking a decision if to accept the tentative composition.

In step 206, the control unit 40 receives a confirmation signal from the UI 50. Step 206 may be the result of the caretaker accepting, via the UI 50, the tentative/−calculated composition that is displayed in step 204. Reverting to FIG. 3C, the confirmation signal may be generated when the user activates the acceptance button (YES) in the screen image 300C. The confirmation signal causes the control unit 40 to configure the FGA 10 to generate the treatment fluid with the tentative composition. As noted in respect of FIG. 1, the control unit 40 may configure the FGA 10 by providing configuration data to a local controller in the FGA 10 or by directly controlling the operation of the FGA 10. If the user instead activates the decline button (NO) in screen image 300C, no confirmation signal is generated, and the FGA 10 is not configured in accordance with the tentative composition.

In some embodiments, the control unit 40 continuously displays the composition of the treatment fluid on the UI 50, for example as shown in FIG. 3C, while the FGA 10 is operated to generate the treatment fluid subsequent to step 206, optionally upon user request via the UI 50. This will allow a caretaker, who may be responsible for many patients in treatment, to quickly be informed about the current composition of the treatment fluid provided for a particular patient.

It may be difficult for a caretaker, especially in a stressful situation, to evaluate if the tentative/calculated composition displayed in step 204 is acceptable and medically safe for the patient. Further, it should be noted that the composition calculated in step 202 is a nominal composition, which is calculated based on the nominal compositions of the concentrate(s) that are to be mixed with water. Because of production tolerances, the concentration of components in a concentrate will deviate from batch to batch. The allowable deviations from nominal values are regulated, for example by pharmacopeia or legal frameworks. The allowable deviations may be defined for the concentrate itself or for the resulting treatment fluid. For example, the concentration of a component in the treatment fluid may be allowed to deviate by ±10%, ±5% or ±2.5% from its nominal concentration. The allowable deviation may differ between components. Given that nominal composition is known for each batch of concentrate, but deviations between batches are not, the tentative composition that is displayed in step 204 may deviate from the actual composition of the treatment fluid to be generated by the FGA 10. This may make it even more difficult for the caretaker to assess if the tentative composition is acceptable and medically safe. These problems are addressed by optional steps 203 and 205 in FIG. 1.

In step 203, the control unit 40 evaluates the concentration value of the respective component as calculated in step 202 in relation to a corresponding concentration range. The concentration range is a “safety range” and is given by lower and upper concentration limits. Within the safety range, the medical risk for the patient is deemed to be low. The safety range may, for example, be defined by a regulatory authority or other organization, or by the individual clinic. The safety range may be generic for all patients or a group of patients, or may be specific to a patient. The control unit 40 may detect a potential medical risk whenever a concentration value of the tentative composition deviates from the safety range.

In step 205, if a potential medical risk is detected in step 203, the control unit 40 displays a corresponding warning message on the UI 50. The warning message may also indicate the specific component that is deemed to pose the potential medical risk. This may be done in many different ways. An example is shown in FIG. 3C, where a warning message 60 is displayed next to the component (here, K⁺). The warning message may also include the safety range that is violated. In another alternative, the safety ranges for all components are displayed and the violated safety range is highlighted.

In step 203, the control unit 40 may also account for the above-mentioned deviations when evaluating the nominal concentration values. Thus, in addition to a nominal concentration value, the control unit 40 may calculate minimum and maximum concentration values in the treatment fluid for a component, given the allowable deviation of the component. The control unit 40 may detect a potential medical risk if the maximum or the minimum concentration value falls outside the safety range, even if the nominal concentration value falls within the safety range.

To give a non-limiting example, consider a dialysis fluid for extracorporeal blood treatment generated by mixing an acid (A) concentrate and a bicarbonate (B) concentrate with water. Assume that the dialysis fluid, when generated with a nominal sodium concentration of 140 mM, has a nominal potassium concentration of 4 mM. If the applicable pharmacopeia prescribes an accuracy of +10% for potassium concentration in the dialysis fluid, the actual potassium concentration in the dialysis fluid will be in the range 3.6-4.4 mM. Assuming that the upper limit of the safety range is 4.6 mM, no medical risk will be identified in step 203. If the caretaker instead wants to generate the dialysis fluid with a nominal sodium concentration of 160 mM, the nominal potassium concentration will be 4.57 mM. However, the actual potassium concentration in the dialysis fluid will be in the range 4.1-5.0 mM. Thus, if step 203 only considers the nominal potassium concentration, a medical risk will not be detected although such a risk may actually exist.

If the lower concentration limit of the safety range for a component is violated, the caretaker may compensate for the resulting deficit by performing a separate infusion of an additional amount of the component into the blood of the patient. It is realized that steps 203, 205 will also serve to raise the awareness of the caretaker about the need for such compensation.

The method 200 may be particularly useful in connection with extracorporeal blood treatment, especially acute blood treatment. Such treatment is commonly performed in critical care settings such as ICUs, by staff that may be under significant stress and may not be experts on extracorporeal blood treatment. Such staff will benefit greatly from the support provided by method 200. For patients with AKI, the RRT arrangement 20 in FIG. 1 is commonly configured for continuous renal replacement therapy (CRRT). A CRRT treatment is performed by a machine 24 hours a day to slowly and continuously clean out waste products and fluid from the patient. CRRT is well-known to the skilled person and will not be described in further detail.

Reverting to step 201 of method 200, the selected component may be any component in the treatment fluid to be generated. In the field of extracorporeal blood treatment, non-limiting examples of the selected component include sodium, bicarbonate, and potassium. In the field of peritoneal dialysis, a non-limiting example of the selected component includes sodium.

Further, the other components for which concentration values are calculated in step 202 may be any or all components that will be present in the treatment fluid to be generated (other than the selected component). Generally, the other components comprise one or more electrolytes. In some embodiments, the other components comprise small ions, for example monatomic ions. Such electrolytes may include one or more of magnesium, potassium, calcium, sodium, or chloride. In some embodiments, the other components comprise polyatomic ions, such as acetate, citrate, lactate, phosphate or bicarbonate. In some embodiments, the other components comprise an osmotic agent such as glucose (aka dextrose), L-carnitine, glycerol, icodextrin, fructose, sorbitol, mannitol or xylitol.

Any commercially available concentrate or combination of two or more concentrates may be used in the FGA 10 to generate the treatment fluid. Below, a few embodiments for generation of dialysis fluid are described. In some embodiments, a dialysis fluid for use in chronic blood treatment is generated by mixing a single concentrate with water at a dilution ratio of 10-50 by volume. In a non-limiting example, the single concentrate comprises lactate, sodium, potassium, calcium, magnesium, glucose and chloride. Such a concentrate is, for example, commercially available for the PureFlow SL system from NxStage. Alternatively, the dialysis fluid may be generated by mixing two concentrates with water. For example, a bicarbonate (B) concentrate and an acid (A) concentrate may be mixed with water at a dilution ratio of 10-50. The bicarbonate concentrate is also known as “base concentrate” and need not contain only bicarbonate. A and B concentrates are commercially available and well-known in the art. In a non-limiting example, the B concentrate comprises bicarbonate, and the A concentrate comprises sodium, potassium, calcium, magnesium, glucose, acetate and chloride. In some A concentrates, acetate is replaced or supplemented by another acid, for example lactate, citrate or hydrochloric acid. In the BiCart Select® system from Baxter, dialysis fluid is generated by mixing three different concentrates, namely a bicarbonate concentrate (BiCart®), a sodium chloride concentrate (SelectCart®), and a concentrate with acid and other electrolytes (SelectBag®). In some embodiments, dialysis fluid for CRRT treatment is generated by mixing at least one concentrate with water. In a non-limiting example, such a dialysis fluid comprises bicarbonate, sodium, potassium, calcium, magnesium, phosphate, glucose, acetate and chloride. In some embodiments, dialysis fluid for use in PD is generated by mixing at least one concentrate with water. Example compositions of PD concentrates, to be mixed with water individually or in combination, are disclosed in US2018/0021501 and WO2017/193069, which are incorporated herein by reference.

FIG. 2B shows the method 200 as performed in the context of an overall operating procedure 210 for the FGA 10 in FIG. 1. The method 210 may be performed by the control unit 40. The operating procedure 210 involves a step 211 in which the FGA 10 is operated to generate treatment fluid with a current composition. At any time during step 211, the control unit 40 performs the method 200, triggered by user interaction via the UI 50. If no confirmation signal is received in step 206 (FIG. 2A), the FGA 10 is maintained in operation to generate the treatment fluid in accordance with the current composition (step 213). If the confirmation signal is received, the procedure 210 is directed by step 212 to step 214, in which the FGA 10 is operated to generate treatment fluid with the tentative/calculated composition that was accepted by the user in step 206. Thereby, the current composition will be changed into the tentative composition.

FIG. 2C shows an example procedure that may be part of step 202 in FIG. 2A. In step 202A, the control unit 40 obtains a concentrate ID for each concentrate that is available in the FGA 10 for use in generation of treatment fluid. In one example, the user manually enters one or more concentrate IDs via the UI 50. Alternatively or additionally, one or more concentrate IDs may be provided to the control unit 40 from an automatic detection system (not shown) in the FGA 10. The detection system may be operable to automatically detect the concentrate ID of a container with concentrate when installed in the FGA 10, for example by detection of text or machine-readable code on the container. In step 202B, the control unit 40 obtains composition data for each of the concentrate IDs. The composition data may designate the nominal composition of the respective concentrate, in terms of included components and their nominal concentrations. For example, the control unit 40 may retrieve the composition data from internal memory (cf. 42 in FIG. 2) or from an external database that is accessible to the control unit 40. In step 202C, which is optional, the control unit 40 obtains constraint data for the mixing of the concentrate(s) with water. The constraint data may, for example, define one or more mixing ratios that cannot be changed, for example that one of plural concentrates must be mixed with water at a predefined ratio, or that two concentrates must be mixed at a predefined ratio. Alternatively or additionally, the constraint data may define allowable ranges for one or more mixing ratios. The control unit 40 may retrieve the constraint data from internal memory (cf. 42 in FIG. 2) or from an external database. In step 202D, the control unit 40 operates a predefined calculation function on the composition data, the constraint data (if obtained) and the candidate set value to determine the tentative composition of the treatment fluid. The tentative composition may, but need not, include concentration values of all components in the treatment fluid to be generated. The calculation function is based on straight-forward relations, and the skilled person can without difficulty derive a calculation function tailored to a specific treatment fluid that is generated from one or more specific concentrates. The calculation function may be implemented by use of one or more predefined look-up tables and/or by runtime evaluation of a mathematical algorithm. It may be noted that the constraint data, if not obtained in step 202C, may be embedded in the calculation function.

Reverting to the method 200 in FIG. 1, it is conceivable that the confirmation step 206 is omitted in some embodiments. This means that the operator is not required or requested to confirm the calculated composition given by step 202. Instead, the control unit 40 automatically configures the FGA 10 to generate the treatment fluid with the calculated composition, for example directly after step 201 when the candidate set value has been input. This type of operational control may be acceptable for at least some types of RRT, for example in chronic blood treatment. By step 204, the operator is made aware of the composition of the resulting treatment fluid, in terms of displayed concentration value(s).

FIG. 4 is included to provide a non-limiting example of an FGA 10. In the illustrated example, the FGA 10 is configured to generate a treatment fluid TF by mixing water 11 with two liquid concentrates 12, 13. The water 11 is held in a container 14A, and the concentrates 12, 13 is held in a respective container 14B, 14C. The containers 14B, 14C may be replaced when empty, whereas the water container 14A is repeatedly replenished from a water source 16. The FGA 10 further comprises a mixing container 14D and a storage container 14E. An infeed line 15A extends from the water source 16 to the mixing container 14D and is connected for fluid communication with the containers 14A, 14B, 14C. A control valve 16A is arranged in the infeed line 15A to control the flow of water from the source, and control valves 17A, 17B, 17C are arranged in connecting lines between the infeed line 15A and the containers 14A, 14B, 14C. A first fluid pump 18A is arranged in the infeed line 15A downstream of the connecting lines. A supply line 15B is arranged to extend between the mixing tank 14D and the storage tank 14E. A recirculation line 15C connects the supply line 15B with the top of the mixing tank 14D. A second fluid pump 18B and a control valve 17D are arranged in the supply line 15B, upstream and downstream of the recirculation line 15C, respectively. An outlet line 15D extends from the storage tank 14E and may be connected to the fluid line 30 in FIG. 1. A third fluid pump 18C is arranged in the outlet line 15D.

The FGA 10 in FIG. 4 may be operated to intermittently replenish the water container 14A by selectively opening valves 16A, 17A, while valves 17B, 17C are closed and pump 18A is stopped. The pump 18A is presumed to be occluding. To generate treatment fluid, valves 17A, 17B, 17C are opened in sequence and pump 18A is activated to pump proportioned amounts of water 11 and concentrates 12, 13 into the mixing container 14D. Pump 18B is activated while valve 17D is closed to recirculate the fluid in the mixing tank 14D to improve mixing of water and concentrates. After a time period, valve 17D is opened and treatment fluid is pumped from the mixing tank 14D into the storage tank 14E. Then, treatment fluid is pumped by pump 18C into the outlet line 15D to provide the treatment fluid to the RRT arrangement (20 in FIG. 2).

The proportions of water and concentrates that are pumped into the mixing tank 14D may be controlled by the use of one or more flow meters and/or one or more conductivity sensors and/or by monitoring the weight of the mixing container 14D and/or the containers 11, 12, 13, as is well-known in the art.

In a variant, the mixing tank 14D, the pump 18B and the recirculation line 15C are omitted, and water 11 and concentrates 12, 13 are pumped into the storage tank 14E for mixing therein. In a further variant, the storage tank 14E and the pump 18C are also omitted, and water 11 and concentrates 12,13 are concurrently proportioned into the infeed line 15A to mix therein, resulting in treatment fluid being provided without intermediate storage.

FIG. 5 is included to provide a non-limiting example of an RRT arrangement 20 that may be used in combination with an FGA 10, for example as shown in FIG. 4. The RRT arrangement 20 may, for example, be used in CRRT. In FIG. 5, the RRT arrangement 20 is connected to a patient P at a blood withdrawal end and a blood return end. The connections may be performed by any conventional device, such as needle or catheter. The RRT arrangement 20 comprises a disposable 21 with blood lines or tubes that define a blood withdrawal path 23 and a blood return path 24. A dialyzer 26 is connected between the withdrawal and return paths 23, 24. A blood pump 22 is arranged to draw blood from the patient P and pump the blood via the blood compartment of the dialyzer 26 and back to the patient P. The dialyzer 26 is connected to receive dialysis fluid on fluid path 29′ and to output effluent on fluid path 29″. In the illustrated example, the RRT arrangement 20 further comprises a first source 27A of replacement fluid which is connected by a fluid line 27B to the withdrawal path 23 intermediate the blood pump 22 and the dialyzer 26. A fluid pump 27C is arranged to pump the replacement fluid from source 27A into the withdrawal path 23. The RRT arrangement 20 further comprises a second source 28A of replacement fluid which is connected by a fluid line 28B to the return path 24. A fluid pump 28C is arranged to pump the replacement fluid from source 28A into the return path 24. In the example of CRRT, the RRT arrangement 20 may also comprise an arrangement for infusion of an anti-coagulant agent, for example citrate or heparin, or an arrangement for infusion of a calcium-containing solution.

It is understood that an FGA 10, for example as shown in FIG. 4, may be connected by a fluid line (30 in FIG. 1) to provide the dialysis fluid to the dialyzer 26 in FIG. 5. Alternatively or additionally, the replacement fluid may be generated by the FGA 10.

As noted, FIG. 5 is merely an example, and the RRT arrangement 20 may include other conventional components, such as clamps, pressure sensors, air detector, etc. Also, the pre-infusion and/or post-infusion of replacement fluid may be omitted.

While the subject of the present disclosure has been described in connection with what is presently considered to be the most practical and preferred embodiments, it is to be understood that the subject of the present disclosure is not to be limited to the disclosed embodiments, but on the contrary, is intended to cover various modifications and equivalent arrangements included within the spirit and the scope of the appended claims.

Further, while operations are depicted in the drawings in a particular order, this should not be understood as requiring that such operations be performed in the particular order shown or in sequential order, or that all illustrated operations be performed, to achieve desirable results. In certain circumstances, parallel processing may be advantageous.

1. A system, comprising:
a fluid generation arrangement, which is operable to mix one or more concentrates with water to generate a treatment fluid for use in renal replacement therapy,
a user interface, and
a control unit, which is connected to the user interface and arranged to configure the fluid generation arrangement, the control unit being configured to:
receive, from the user interface, a candidate set value of a selected component of the treatment fluid,
calculate, for the candidate set value, a calculated composition of the treatment fluid so that the calculated composition includes the selected component at a concentration that matches the candidate set value, and
display, on the user interface, a respective concentration value of one or more components other than the selected component in the calculated composition.

2. The system of claim 1, wherein the control unit is further configured to configure, upon receipt of a confirmation signal from the user interface, the fluid generation arrangement to generate the treatment fluid in accordance with the calculated composition.

3. The system of claim 2, wherein the control unit is operable to receive the candidate set value, calculate the calculated composition, and display the respective concentration value while the fluid generation arrangement is operated to generate the treatment fluid with a current composition which differs from the calculated composition, and wherein the control unit is configured to, in absence of the confirmation signal, maintain the fluid generation arrangement in operation to generate the treatment fluid in accordance with the current composition.

4. The system of claim 1, wherein the one or more concentrates comprises a combination of an acid concentrate and a bicarbonate concentrate.

5. The system of claim 1, wherein the selected component comprises one of sodium, bicarbonate or potassium.

6. The system of claim 1, wherein the one or more components other than the selected component comprises one or more electrolytes.

7. The system of claim 6, wherein the one or more electrolytes is one or more of magnesium, potassium, calcium, sodium, chloride, phosphate, bicarbonate, lactate, acetate, citrate.

8. The system of claim 1, wherein the one or more components other than the selected component comprises an osmotic agent.

9. The system of claim 1, wherein the one or more components other than the selected component comprises glucose.

10. The system of claim 1, wherein the control unit is further configured to: obtain nominal composition data of the one or more concentrates, wherein the control unit is configured to calculate the calculated composition as a function of the nominal composition data, so that the candidate set value is achieved in the calculated composition.

11. The system of claim 1, wherein the control unit is further configured to: evaluate concentrations of components in the calculated composition in relation to a respective concentration range, and display a warning message on the user interface if at least one of the components has a concentration that deviates from the respective concentration range.

12. The system of claim 11, wherein the control unit is configured to display the warning message so as to indicate the at least one of the components on the user interface.

13. The system of claim 11, wherein the concentrations of the components comprise nominal concentrations, wherein the control unit is configured to calculate the nominal concentrations based on a nominal composition of a respective concentrate among the one or more concentrates.

14. The system of claim 11, wherein the concentrations of the components comprise at least one of a maximum concentration or a minimum concentration of a respective component in the calculated composition, wherein the control unit is configured to calculate the at least one of the maximum concentration or the minimum concentration based on an allowable deviation from a nominal concentration in the treatment fluid, and wherein the control unit is further configured to display the warning message if the at least one of the maximum concentration or the minimum concentration deviates from the concentration range.

15. The system of claim 1, further comprising a treatment arrangement for the renal replacement therapy, wherein the control unit is further configured to operate the fluid generation arrangement to generate the treatment fluid in accordance with the calculated composition, and to operate the treatment arrangement to perform the renal replacement therapy by use of the treatment fluid generated by the fluid generation arrangement.

16. The system of claim 15, wherein the treatment arrangement is configured to perform extracorporeal blood treatment.

17. The system of claim 15, wherein the treatment arrangement is configured to perform treatment of acute kidney injury.

18. The system of claim 15, wherein the treatment arrangement is configured to perform peritoneal dialysis.

19. The system of claim 1, wherein the control unit is configured to automatically, upon receiving the candidate set value from the user interface, configure the fluid generation arrangement to generate the treatment fluid (TF) in accordance with the calculated composition.

20. A computer-implemented method of configuring a fluid generation arrangement, which is operable to mix one or more concentrates with water to generate a treatment fluid, the computer-implemented method comprising:
receiving, from a user interface, a candidate set value of a selected component of the treatment fluid;
calculating, for the candidate set value, a calculated composition of the treatment fluid so that the calculated composition includes the selected component at a concentration that matches the candidate set value; and
displaying, on the user interface, a respective concentration value of one or more components other than the selected component in the calculated composition.

21. The computer-implemented method of claim 20, further comprising: configuring, upon receipt of a confirmation signal from the user interface, the fluid generation arrangement to generate the treatment fluid in accordance with the calculated composition.

22. The computer-implemented method of claim 20, further comprising: configuring, upon receiving the candidate set value, the fluid generation arrangement to generate the treatment fluid in accordance with the calculated composition.

23. A computer-readable medium comprising computer instructions which, when executed by a control unit in a system according to claim 1, causes the control unit to perform the method of claim 20.
